(19) United States
(12) Patent Application Publication
(10) Pub. No.: US 2004/0137610 A1
(43) Pub. Date: Jul. 15, 2004

(54) Biofilter system equipped with inlet load equalizer for removing volatile organic compounds

(76) Inventors: Yong-Seok Park, Ulsan (KR); Yeal-Soon Hwang, Ulsan (KR); Hyung-Charn Kim, Ulsan (KR); Yong-Taek Yi, Ulsan (KR)

Correspondence Address: Abelman Frayne & Schwab, 150 East 42nd Street, New York, NY, 10017-5612, US

(21) Appl. No.: 10/474,867

(22) PCT Filed: Apr. 12, 2002
(86) PCT No.: PCT/KR02/00673

(30) Foreign Application Priority Data
Apr. 20, 2001 (KR) 200121406

(51) Int. Cl. B01D 53/85; C12M 1/36

(52) U.S. Cl. 435/299.1; 435/286.5

(57) ABSTRACT

Disclosed is a biofilter system for removing volatile organic compounds, generated from various industrial facilities, such as petrochemical plants, refining plants and paint plants, and environmental facilities including sewage treatment plants. The biofilter system comprises a volatile organic compound (VOC) load equilizing portion, a VOC-containing contaminated air transferring portion, a biofiltering portion, and a storing portion for adjusting pH and nutrients in the biofiltering portion. By using such system, an offensive odor and VOC evolved from various industrial facilities and environmental facilities can be efficiently controlled. In addition, even in production processes and storage facilities at which high VOC concentration regulating materials are discontinuously discharged, VOC can be effectively removed.

### TECHNICAL FIELD

The present invention pertains, in general, to a biofilter system equipped with an inlet load-equalizer for removing volatile organic compounds. More specifically, the present invention is directed to a biofilter system for biologically removing high concentrations of volatile organic compounds (for example, benzene, toluene, ethylbenzene, xylene, styrene and so on) discharged from various industrial facilities, such as petrochemical plants, refining plants and paint plants, and environmental facilities including sewage treatment plants.

### PRIOR ART

Generally, volatile organic compounds (VOC) which are produced from various industrial facilities, such as petrochemical plants, refining plants, paint plants and so on, are readily evaporated into the atmosphere due to their high vapor pressure. Photochemical reactions between such evaporated compounds and nitrogen oxides in the atmosphere cause photochemical smog, thereby degrading the ozone layer in the atmosphere. Furthermore, VOC are very toxic to the human body. Starting in 1963, a new atmospheric purification law restricted uses of VOC in the United States. Such restrictions have been mainly enforced in the advanced countries such as United States, Japan and Europe. In Korea, restrictions on use of odorous materials and VOC discharge facilities started from 1995. Nowadays, VOC is more and more strictly regulated so that allowable discharge amount thereof is trending downward.

Various research on removal of an offensive odor and VOC has been performed. Conventionally, there has been adopted physical and chemical methods including activated carbon adsorption, cooling condensation, catalyst combustion, direct combustion, liquid chemical wash and the like. However, such conventional methods suffer from the disadvantages of very high operation cost, dangers such as fire, and generation of secondary contaminants. Therefore, biofilter technologies, which are environment-friendly, have been recently developed to overcome the aforementioned drawbacks and are used in various applications.

In this regard, U.S. Pat. No. 5,869,323 discloses an arrangement for air purification, in which the arrangement includes at least one bioreactor bed, through which air to be purified passes. Furthermore, U.S. Pat. No. 5,891,711 refers to a microbial apparatus for removal of VOC, including a multilevel biofilter which holds a biologically active media at each level, in which the biofilter includes a perforated support plate having a plurality of holes, which prevents the bioactive media from passing therethrough and purifies contaminated fluids therethrough.

Furthermore, Korean Pat. No. 267632 refers to a method for removing an offensive odor and volatile organic compounds comprising the steps of primarily removing offensive odor and volatile materials at an efficiency of 90% or higher by a load equalizer such as an activated carbon, for use in lowering contaminant concentrations, pre-treating offensive odor and volatile materials by a cooling or heating system and a humidification system, continuously providing the pre-treated offensive odor and volatiles by a pressure difference-using pan, and passing such materials through a biological filter with microbial carrier. But, any proper apparatus for removing VOC is not described in this patent.

Korean Pat. Laid-open No. 98-82118 discloses a VOC removing apparatus consisting of a water jacket-equipped reactor in which pall rings are filled, with predetermined amounts of microorganisms forming biofilms on the surface of the pall rings. Korean Pat. Laid-open No. 2000-60699 refers to an offensive odor and VOC removal system which includes a mesh, a porous material, a carbon filter and a nozzle for spraying microorganism culture solution downwards. A biofilter system including a nozzle for spraying microorganism culture media over the carrier layer is also described in Korean Pat. Laid-open No. 2000-12740.

However, such conventional biofilter techniques have the following disadvantages and thus are restricted in their applications.

First, since microorganisms respire and proliferate with the use of inflowing VOC as carbon sources, air stream passage between carriers becomes narrow by overgrowth of microorganisms over time. So, pressure loss and air drift are induced, reducing VOC treatment efficiency. Second, because most of conventional biofilters can be used only under conditions that stream of influent air is constant and concentrations of inflowing VOC are invariably maintained at a predetermined level or less, such biofilter cannot be applied when high concentrations of VOC are discontinuously discharged by repeated influx and efflux during operation of VOC regulating material storage tank. A time period from 10 seconds to several minutes is required to treat VOC with microorganisms based on the concentrations of inflowing VOC. Since a air containing high concentrations of VOC is produced when contents are introduced into the storage tank, the biofilter suitable for use in treatment of such VOC should be fabricated on a large scale. On the other hand, until the next introduction into the storage tank, the gas containing relatively very small amount of VOC is produced. Accordingly, excessively large biofilter results in low load, and thus physiological activity of microorganisms is lowered. In the case of manufacturing the biofilter on a large scale considering high loads of VOC, problems including investment cost and an establishment site of factory occur. Meanwhile, in the case of preparing the biofilter on a small scale considering low load of VOC, high loads of VOC present on influx are not treated and are discharged. Accordingly, with a view to avoiding such situation, a method including a step of pre-treating VOC is performed, but it suffers from poor results.

Thus, the above two problems should be urgently overcome in order to apply biofilters to various industrial facilities.

### DISCLOSURE OF THE INVENTION

Leading to the present invention, the intensive and thorough research on treatment of high concentrations of VOC, carried out by the present inventors aiming to avoid the problems encountered in the prior arts, resulted in the finding a biofilter system equipped with a load-equalizer having a specific configuration for ease of application under the condition of being discontinuously fed VOC of high concentrations, in which by use of a porous microbial carrier maximizing surface area required for growth of microorganisms and for smooth stream of air, and a unit capable of physically shaking and rearranging the microbial carrier, drift of contaminated air caused by unequal growth of microorganisms can be prevented, thereby increasing removal efficiency of VOC.

Therefore, it is an object of the present invention to provide a biofilter system for removing VOC, which is advantageous in terms of ease of application during discontinuous discharge of high concentrations of VOC.

It is another object of the present invention to provide a biofilter system which is capable of preventing decrease of VOC treatment efficiency by air drift and pressure loss due to overgrowth of microorganisms.

It is further object of the present invention to provide a biofilter system which shows stable treatment efficiency even after long term operation.

In accordance with an embodiment of the present invention, there is provided a biofilter system for removing VOC comprising,

a) a load-equalizer comprising a porous carrier layer, an inlet for VOC-containing air positioned under the porous carrier layer, a storage tank for load-equalizing solvent, a circulation unit equipped with a spray nozzle which is connected to the storage tank and sprays the load-equalizing solvent over the carrier layer, said carrier layer being arranged in such a manner that the VOC-containing air contacts with the load-equalizing solvent through the carrier layer to provide a load-equalized air with the VOC concentration range treatable in the subsequent biofiltering by physical transfer of VOC, and an outlet for discharging the load-equalized air;

b) an air transferring portion for introducing the VOC-containing air from VOC generation sources to the load-equalizer and transferring the load-equalized air to a biofiltering portion,

c) a biofiltering portion, comprising a porous microbial carrier layer inhabited by microorganisms for removing VOC, an inlet for the load-equalized air positioned under the porous microbial carrier layer, a medium storage tank and a medium circulation unit, said microbial carrier layer equipped with a pressurized air/water spray unit for shaking and rearranging the microbial carrier layer, thereby suppressing pressure loss and drift of the load-equalized air, said medium circulation unit being connected to the medium storage tank and circulating the medium to two directions by a circulatory pump, in which one direction flows to the biofiltering portion through a upper spray nozzle located above the microbial carrier layer and the other direction flows over the medium through a lower spray nozzle placed under the microbial carrier layer, and

d) a storing portion for adjusting pH of the medium and feeding nutrients, connected to the medium storage tank.

### BRIEF DESCRIPTION OF THE DRAWINGS

FIG. 1 is a schematic diagram of a biofilter system including a load-equalizer and a biofiltering portion according to one embodiment of the present invention.

FIG. 2a is a schematic diagram of a one-stage load-equalizer according to the present invention, and FIG. 2b is a schematic diagram of a two-stage load-equalizer according to the present invention.

FIGS. 3a and 3b are a schematic diagram of a biofilter system including a pressurized air and water spray unit and a medium circulation unit according to the present invention.

FIG. 4 is a graph showing performances of load-equalization and removal of styrene monomer discontinuously discharged from a styrene monomer storage tank by the biofilter system according to the present invention.

FIG. 5 is a graph showing performances of load-equalization and removal of toluene discontinuously discharged from a toluene storage tank by the biofilter system according to the present invention.

FIG. 6 is a graph showing performances of load-equalization and removal of xylene discontinuously discharged from a para-xylene storage tank by the biofilter system according to the present invention.

FIG. 7 is a graph showing performances of load-equalization and removal of methylethylketone discontinuously discharged from a methylethylketone storage tank by the biofilter system according to the present invention.

FIG. 8 is a graph showing performances of load-equalization and removal of benzene discontinuously discharged from a benzene storage tank by the biofilter system according to the present invention.

### BEST MODES FOR CARRYING OUT THE INVENTION

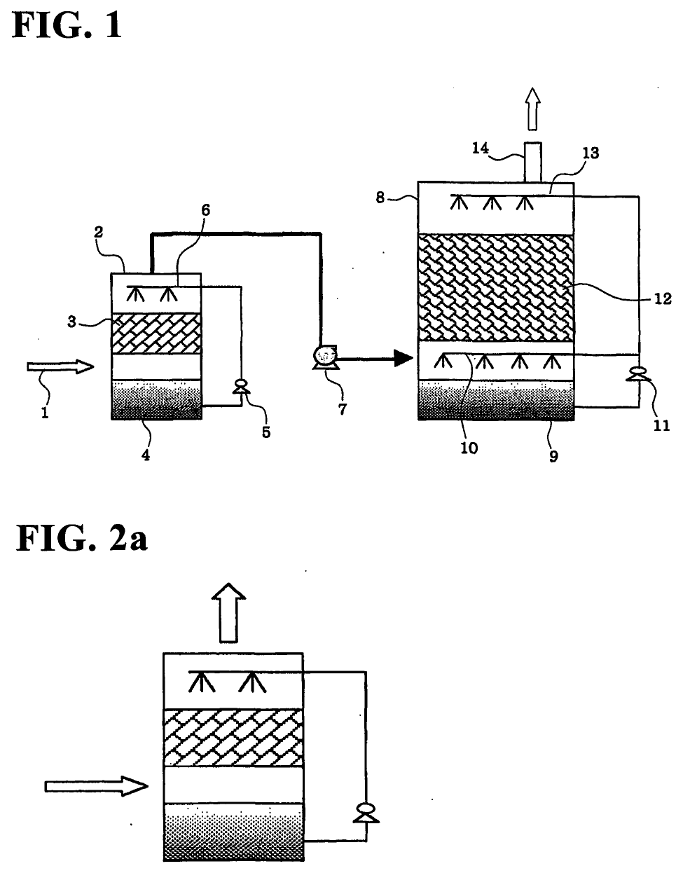

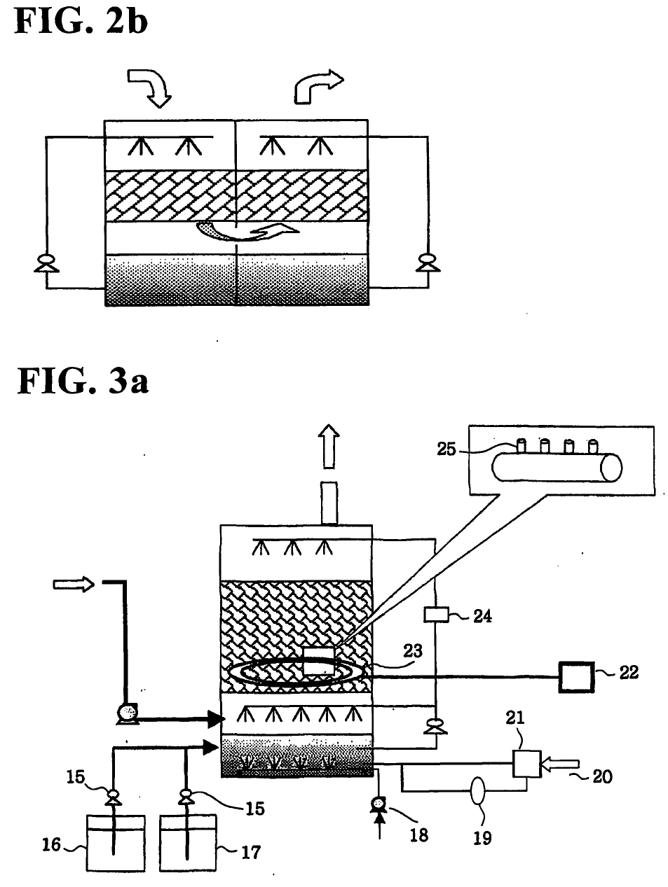

With reference to FIG. 1, there is schematically shown the inventive biofilter system, in which a load-equalizer that allows an air containing high concentrations of VOC introduced discontinuously to be discharged continuously at low concentrations is mounted to the front of the system. FIGS. 2a and 2b shows embodiments of the load-equalizer.

As can be seen in FIG. 1, a load-equalizer 2 in the biofilter system according to one embodiment of the present invention comprises porous carrier layer 3, a VOC-containing air inlet placed under the porous carrier layer, a load-equalizing solvent storage tank 4, a load-equalizing solvent circulation unit, and a load-equalized air outlet placed above the porous carrier layer. The circulation unit is equipped with a circulatory pump 5 and a spray nozzle of the load-equalizing solvent 6.

Generally, VOC-containing air is discontinuously produced when VOC is introduced into storage facilities such as VOC storage tanks or discharged therefrom. The amounts of VOC produced upon influx drastically differ from the ones on efflux. As aforementioned, the high concentrations of VOC is produced when contents are introduced into the storage tank, and then the VOC concentration is relatively very small until the next introduction into the VOC storage tank.

According to the present invention, high concentrations of VOC, which is discharged from the upper part of the VOC storage tank on storing VOC, is absorbed by use of a load-equalizing solvent with a high boiling point in the load-equalizer 2. As a result, the concentration of VOC to be treated by the biofilter is constantly maintained in the specific range (preferably, less than 1000 ppm) during the introduction intervals of the VOC into the storage tank.

The load-equalizing solvent in the storage tank 4 is circulated by the circulatory pump 5. While the VOC-absorbed solvent sprayed from the upper nozzle passes through the carrier layer, the VOC absorbed in the solvent contacts with an influent VOC-containing air, and thus becomes gaseous again, after which such VOC flows into the biofilter, along with the VOC-containing air having passed through the carrier. In other words, when the VOC-containing air comes into contact with the load-equalizing solvent circulated by the circulation unit in the porous carrier layer, VOC is physically transferred from the VOC-containing air to the load-equalizing solvent or vice versa, depending on the concentration of VOC introduced into the load-equalizer.

The carrier layer in the load-equalizer has porous structures prepared with at least one material selected from the group consisting of polyethylene, polypropylene, polyester and ceramic. In addition, the size of the carrier layer which contacts with the influent VOC-containing air ranges from 10% to 50% of volume of the load-equalizing solvent. When below 10%, the liquid/gas ratio (L/m³) is very small, and thus absorption efficiency is reduced. On the other hand, in case of exceeding 50%, the liquid/gas ratio is too large, which causes the increase of the size of the carrier layer.

As for the load-equalizing solvent in the present invention, a lipid-soluble solvent and a water-soluble solvent according to properties of inflowing VOC may be used alone or in combinations thereof. The suitable lipid-soluble solvent comprises C₁₄₋₁₅ paraffin and naphthene hydrocarbons, which is a colorless and odorless liquid, without any ozone layer-damaging components, and is non-aromatic solvent having 0.8 to 0.9 g/cm³ specific gravity, 270 to 320° C. boiling point, 110 to 140° C. ignition point and aromatic components of below 0.5%, or comprises silicon oil having a specific gravity of 0.76 to 1.00 g/cm³, viscosity of 0.65 to 10,000 cSt (25° C.), flow point of −75 to −40° C., and surface tension of 20.0 to 25.5 dyne/cm. The above hydrocarbon-based solvent comprises 60-70% paraffin and 30-40% naphthene. Additionally, the solvent has a load-equalizing capacity constant k in the specific ranges, depending on kinds of VOC. The load-equalizing capability constant k is defined as the ratio between gas concentration of VOC in a headspace and liquid concentration of VOC in the solvent, when VOC is absorbed in the solvent in a closed container and then reaches equilibrium at room temperature. That is to say, k is ‘gas concentration (mg/Nm³)/liquid concentration (mg/L)’. The lower the value of k, the higher the load-equalizing effect by the solvent. In this regard, k of the lipid-soluble solvent ranges from 0.005 to 0.6 for single benzene ring compounds such as benzene, toluene, xylene, ethylbenzene and styrene monomer.

In addition, the water-soluble solvent mainly comprises water and may be additionally added with propyleneglycol, viscosity enhancer and cryoprotective compound. More specifically, k of the water-soluble solvent for methanol and methylethylketone, water-soluble VOC, ranges from 0.001 to 0.1.

As can be seen in FIGS. 2a and 2b, the load-equalizer can be designed with 2 stages or more, with the intention of maintaining proper concentrations of VOC to be streamed into the biofilter, depending on the generation periods and the concentrations of VOC.

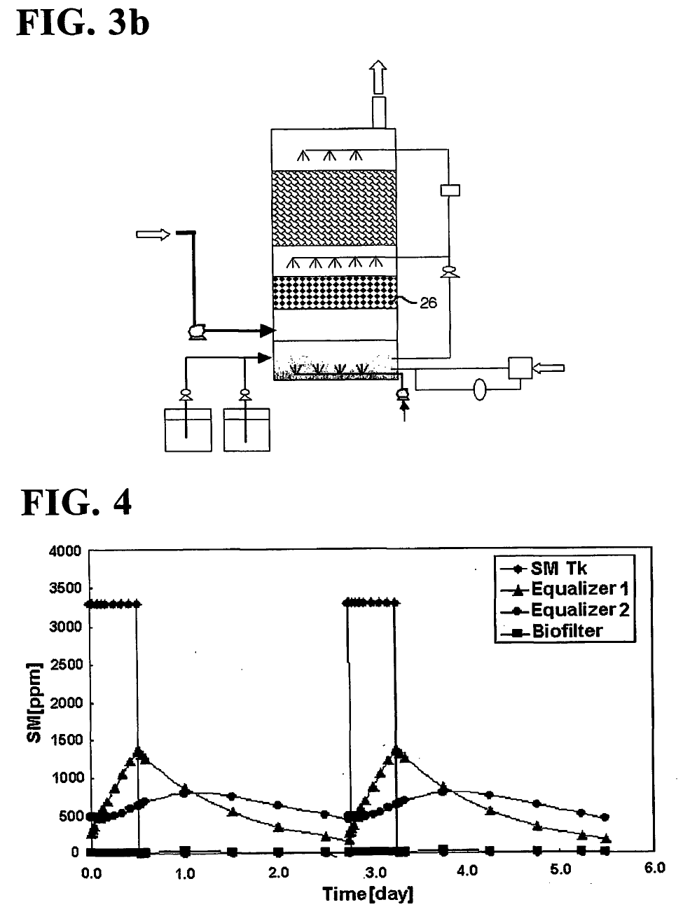

FIGS. 3a and 3b illustrate one embodiment of the inventive biofiltering portion which includes a pressurized air/water spray unit and a medium circulation unit mounted into the porous carrier layer. Referring to FIGS. 1 and 3a, the carrier-charged biofilter system according to the present invention contains a load-equalizer installed to its front for allowing the concentration of VOC in the contaminated air to be adjusted. Further, the biofilter system comprises an air transferring portion including a blower 7, which serve to introducing the contaminated air from VOC generation source to the load-equalizer and to transfer the load-equalized air to the biofiltering portion, a porous microbial carrier layer 12 inhabited by microorganisms for removing VOC, a medium storage tank 9 and a circulation unit for feeding nutrients and water to the microbial carrier layer, a pressurized air/water spray unit 25 for separating excess microorganisms from the carrier layer and blocking drift of the load-equalized air by inducing a uniform growth of microorganisms, a nutrient storage tank 16 for feeding high concentrations of nutrients to the medium storage tank in the predetermined amounts, and a pH adjusting solution storage tank 17 for storing acidic and alkaline nutrients used to adjust the acidity.

Meanwhile, added may be a temperature controller 19 for maintaining a suitable temperature of the medium storage tank in the winter seasons, a water-level controller (not shown) for maintaining a water level of the medium storage tank in the biofilter, and a demister (not shown) before an outlet for removing moisture in the air purified through the biofilter.

According to the embodiment shown in FIGS. 1 and 3a, the mainframe 8 of the biofiltering portion can be made of stainless steel or FRP. The size of the medium storage tank 9, which is positioned in the bottom of the biofiltering portion, is determined in the range of 10-30% of volume of the microbial carrier layer 12. The medium in the medium storage tank is circulated in two directions by use of the circulatory pump 11. In one direction, the medium is fed to the medium storage tank through the lower spray nozzle 10 to circulate the medium. The spray coverage is equal to the total area of the surface of medium in the storage tank, and the medium is circulated continuously. The reason why the medium is circulated is that inflowing contaminated air comes into contact with the medium, whereby VOC dissolved in the medium are directly decomposed by microorganisms in the medium storage tank. As such, with a view to contacting the load-equalized air and the medium, a packing layer 26, which is charged with pall rings in the form of porous cylinders having a diameter and a height of 0.5-2.0 inches, respectively, and made of polyethylene or polypropylene, can be mounted below the lower spray nozzle 10, as can be seen in FIG. 3b.

On the other hand, in the other direction, the medium is sprayed to the carrier layer through the upper medium spray nozzle 13. Spray through the upper nozzle provides water and nutrients, such as nitrogen and phosphorous, to the VOC removing microorganisms living in the carrier layer. The spray nozzle in the medium circulation unit may be operated according to the controllable time period with use of a solenoid valve 24.

In addition, a thermometer, a pH meter and a pH regulator may be mounted to the medium storage tank 9. Acid/alkali for adjusting pH and nutrients are supplied from a pH adjusting solution storage tank 17 and a nutrient storage tank 16, respectively, by quantitative pumps 15.

Microorganisms which can effectively remove VOC live in the microbial carrier layer of the biofiltering portion. Such a carrier layer is prepared by fixing at least one microorganism selected from the group consisting of Pseudomonas, Aerobacter, Bacillus, Microbacterium and Arthrobacter Sp., to the porous carrier made of a material selected from the group consisting of polyether, polyester and polyethylene. The microbial carrier layer is 0.5-2 m high, and two or more layers may be provided depending on the load of VOC in inflowing air.

The pressurized air/water spray unit 23 which is installed in the lower microbial carrier layer sprays the pressurized air/water upwards by a spray nozzle 25 facing upwardly, whereby the microbial carrier layer is shaken and rearranged, and thus pressure loss and drift of the load-equalized air caused by excess microorganisms can be prevented. For this purpose, the biofiltering portion additionally includes a compressor 22 and a controller for controlling the spray unit, which allows the pressurized air/water to be automatically sprayed when pressure loss on introduction of the load-equalized air to the microbial carrier layer reaches a predetermined value, for instance, 200 mmH₂O.

In order to maintain activities of microorganisms in the biofiltering portion, the temperature suitable for living microorganisms should be maintained. Hence, the temperature controller 19 is connected to the medium storage tank to maintain the temperature of the carrier layer on the predetermined level (i.e., 20° C. or higher). As such, steam and electricity can be used as heat sources.

For the facilitation of decomposition of VOC by microorganisms present in the medium storage tank, air is provided to the lower part of the medium storage tank through a ring blower 18. This is designed so that the medium storage tank serves as an aeration tank.

A better understanding of the present invention may be obtained in light of the following examples which are set forth to illustrate, but are not to be construed to limit the present invention.

### EXAMPLE 1

Load-Equalizing Effects and Removal of Styrene Monomer Discontinuously Discharged from Styrene Monomer Storage Tank

The biofilter system including the load-equalizer and the biofiltering portion was operated under conditions shown in the following table 1. The load-equalizing effects and removal of styrene monomer were analyzed. The results were presented in FIG. 4.

TABLE 1
Specification of Styrene Monomer (SM) Storage
Tank and Operation Data Thereof

| | | Unit |
|---|---|---|
| Capacity of SM Storage Tank | 6615 | m³ |
| Flowing Volume of SM per Month | 30000 | m³/month |
| SM Influx No. | 11 | no./month |
| Influx Period | 2.73 | Day |
| Volume of SM Streamed in SM Storage | 2800 | m³ |
| Tank per Influx | | |
| Influx Time | 11.2 | Hr |
| Influx Rate | 5.0 | m³/min |
| Concentration of SM Discharged from | 6580 | Ppm |
| SM Storage Tank on Influx | | |

In the case of sucking the contaminated air from the SM storage tank at 10 m³/min using the blower under operating conditions described in the above table, the concentration of styrene monomer in the gas flowing in the load-equalizer was about 3,290 ppm (at 18° C.) at influx and then 0 ppm before the next influx. High concentration of styrene monomer inflowing discontinuously as above was decreased to less than 700 ppm by the load-equalizer with two stages, each of which had a load-equalizing solvent volume of 2 m³ (lipid-soluble load-equalizing solvent Qvesol-O (supplied from Q-BioTech Corp.)). Then, the styrene monomer was diluted with atmospheric air and streamed in the biofilter at 500 ppm. By the load-equalization, stable treatment efficiency of 95% or more could be obtained even through the small biofilter. Therefore, the styrene monomer contained in the air can be treated by the biofilter having a microbial carrier layer of 14 m³ by use of the load-equalizer under the above conditions.

### COMPARATIVE EXAMPLE 1

Comparative Example 1 was carried out under the same conditions as in Example 1, except that the biofilter system with no load-equalizer was used. As a result, a biofilter having a microbial carrier layer of 70 m³, about 5 times the size of the microbial carrier layer in Example 1, was required to treat the VOC discharged upon influx of styrene monomer.

### EXAMPLE 2

Load-Equalizing Effects and Removal of Toluene Discontinuously Discharged from Toluene Storage Tank

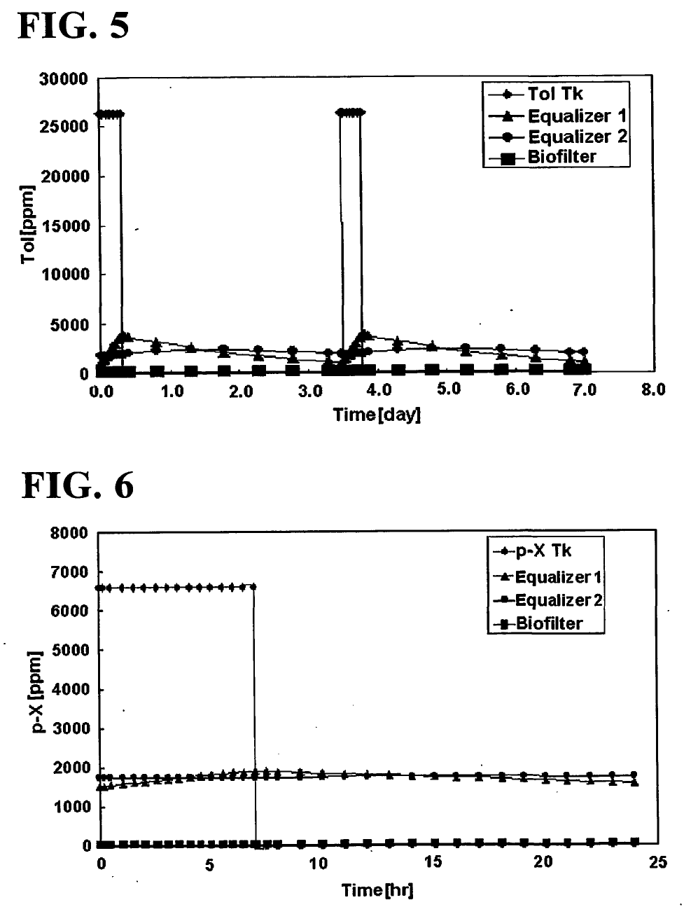

The biofilter system including the load-equalizer and the biofiltering portion was operated under conditions shown in the following table 2. The load-equalizing effects and removal of toluene were analyzed. The results are given in FIG. 5.

TABLE 2
Specification of Toluene Storage
Tank and Operation Data Thereof

| | | Unit |
|---|---|---|
| Capacity of Toluene Storage Tank | 2000 | m³ |
| Flowing Volume of Toluene per Day | 1600 | m³/day |
| Toluene Influx No. | 2 | no./week |
| Toluene Influx Period | 3.5 | day |
| Volume of Toluene Streamed in Toluene | 1600 | m³ |
| Storage Tank per Influx | | |
| Influx Time | 6.4 | hr |
| Influx Rate | 4.2 | m³/min |
| Concentration of Toluene Discharged | 28060 | ppm |
| from Toluene Storage Tank on Influx | | |

In the case of sucking the contaminated air at 4.5 m³/min using the blower under operating conditions described in the above table, concentration of toluene flowing in the load-equalizer was about 26,190 ppm (at 19.3° C.) at influx and then 0 ppm before the next influx. High concentration of toluene inflowing discontinuously as above was decreased to less than 2400 ppm by the load-equalizer with two stages, each of which had a load-equalizing solvent volume of 2 m³ (lipid-soluble solvent Qvesol-O (supplied from Q-BioTech Corp.)). Then, toluene was diluted with atmospheric air and streamed in the biofilter at 500 ppm.

By the load-equalization, stable treatment efficiency of 95% or more could be obtained even through the small biofilter. As such, the used biofilter had a microbial carrier layer of 11 m³.

### COMPARATIVE EXAMPLE 2

Comparative Example 2 was carried out in the same condition as in Example 2, except that the biofilter system with no load-equalizer was used. As a result, the biofilter having the microbial carrier layer of 120 m³, about 10 times the size of the layer used in Example 2, was required to treat the VOC discharged upon influx of toluene.

### EXAMPLE 3

Load-Equalizing Effects and Removal of Para Xylene Discontinuously Discharged from Para Xylene Storage Tank

The biofilter system including the load-equalizer and the biofiltering portion was operated under conditions shown in the following table 3. The load-equalizing effects and removal of para xylene were analyzed. The results are shown in FIG. 6.

TABLE 3
Specification of Para Xylene (p-X) Storage
Tank and Operation Data Thereof

| | | Unit |
|---|---|---|
| Capacity of p-X Storage Tank | 1800 | m³ |
| Flowing Volume of p-X per Day | 1000 | m³/day |
| p-X Influx No. | 1 | no./day |
| p-X Influx Period | 1 | Day |
| Volume of p-X Streamed in p-X Storage | 1000 | m³ |
| Tank per Influx | | |
| Influx Time | 6.7 | Hr |
| Influx Rate | 2.5 | m³/min |
| Concentration of p-X Discharged from | 7890 | Ppm |
| p-X Storage Tank on Influx | | |

In the case of sucking the contaminated air at 3 m³/min using the blower under operating conditions as in the above table, concentration of para xylene flowing in the load-equalizer was about 6,580 ppm (at 17.8° C.) at influx and then 0 ppm before the next influx. High concentration of para-xylene inflowing discontinuously as above was decreased to less than 1,800 ppm by the load-equalizer with two stages, each of which had load-equalizing solvent volume of 2 m³ (lipid-soluble solvent Qvesol-O (supplied from Q-BioTech Corp.)). Then, para xylene was diluted with atmospheric air and streamed in the biofilter at 500 ppm.

By the load-equalization, stable treatment efficiency of 95% or more could be obtained even through the biofilter of small size. The used biofilter had a microbial carrier layer of 6 m³.

### COMPARATIVE EXAMPLE 3

Comparative Example 3 was carried out under the same conditions as in Example 3, except that the biofilter system with no load-equalizer was used. As a result, the biofilter having the carrier layer of 20 m³, about 6 times the size of the layer in Example 3, was required to treat VOC discharged upon influx of para xylene.

### EXAMPLE 4

Load-Equalizing Effects and Removal of Methylethylketone (MEK) Discontinuously Discharged from Methylethylketone Storage Tank

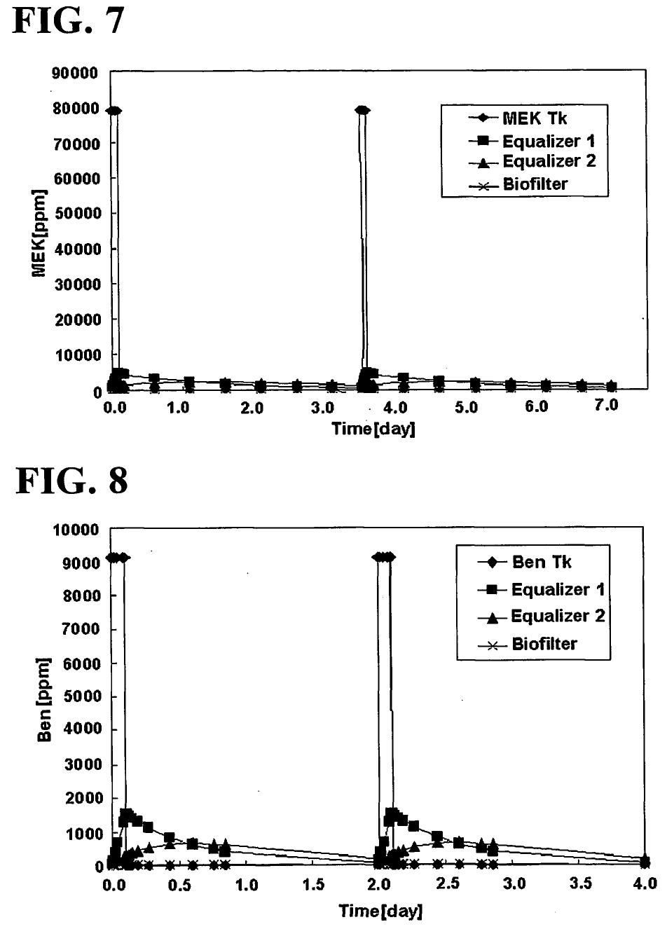

The biofilter system including the load-equalizer and the biofiltering portion was operated under conditions shown in the following table 4. The load-equalizing effects and removal of methylethylketone were analyzed. The results are given in FIG. 7.

TABLE 4
Specification of Methylethylketone (MEK)
Storage Tank and Operation Data Thereof

| | | Unit |
|---|---|---|
| Capacity of MEK Storage Tank | 500 | m³ |
| MEK Influx No. | 2 | No./day |
| MEK Influx Period | 3.5 | Day |
| Volume of MEK Streamed in MEK Storage | 400 | m³ |
| Tank per Influx | | |
| Influx Time | 2.0 | Hr |
| Influx Rate | 3.3 | m³/min |
| Concentration of MEK Discharged from | 83700 | Ppm |
| MEK Storage Tank on Influx | | |

In the case of sucking the contaminated air at 3.5 m³/min using the blower under operating conditions described in the above table, concentration of methylethylketone flowing in the load-equalizer was about 78,920 ppm (at 15° C.) at influx and then 0 ppm before the next influx. High concentration of methylethylketone inflowing discontinuously as above was reduced to less than 2,500 ppm by the load-equalizer with two stages, each of which had load-equalizing solvent volume of 2 m³ (water-soluble solvent Qvesol-W (supplied from Q-BioTech Corp.)). Then, methylethylketone was diluted with atmospheric air and streamed in the biofilter at 500 ppm.

By the load-equalization, stable treatment efficiency of 95% or more could be obtained even through the small biofilter. The used biofilter had a microbial carrier layer of 15 m³.

### COMPARATIVE EXAMPLE 4

Comparative Example 4 was carried out under the same conditions as in Example 4, except that the biofilter system with no load-equalizer was used. As a result, the biofilter having the carrier layer of 150 m³, about 10 times the size of the layer in Example 4, was required to treat VOC discharged upon influx of methylethylketone.

### EXAMPLE 5

Load-Equalizing Effects and Removal of Benzene Discontinuously Discharged from Benzene Storage Tank

The biofilter system including the load-equalizer and the biofiltering portion was operated under conditions shown in the following table 5. The load-equalizing effects and removal of benzene were analyzed. The results are shown in FIG. 8.

TABLE 5
Specification of Benzene Storage
Tank and Operation Data Thereof

| | | Unit |
|---|---|---|
| Capacity of Benzene Storage Tank | 2700 | m³ |
| Benzene Influx No. | 0.5 | no./day |
| Benzene Influx Period | 2 | day |
| Volume of Benzene Streamed in Benzene | 480 | m³ |
| Storage Tank per Influx | | |
| Influx Time | 2.4 | hr |
| Influx Rate | 3.5 | m³/min |
| Concentration of Benzene Discharged | 26320 | ppm |
| from Benzene Storage Tank on Influx | | |

In the case of sucking the contaminated air at 10 m³/min using the blower under operating conditions described in the above table, concentration of benzene flowing in the load-equalizer was about 9,210 ppm (at 32° C., benzene tank is an internal floating roof tank (IFRT)) at influx and then 0 ppm until the next influx. High concentration of benzene inflowing discontinuously as above was reduced to less than 700 ppm by the load-equalizer with two stages, each of which had load-equalizing solvent volume of 2 m³ (lipid-soluble solvent Qvesol-O (supplied from Q-BioTech Corp.)). Then, benzene was diluted with atmospheric air and streamed in the biofilter at 500 ppm.

By the load-equalization, stable treatment efficiency of 95% or more could be obtained even through the small biofilter. The biofiler had a microbial carrier layer of 20 m³.

### COMPARATIVE EXAMPLE 5

Comparative Example 5 was carried out under the same conditions as in Example 5, except that the biofilter system with no load-equalizer was used. As a result, the biofilter having the carrier layer of 150 m³, about 7 times the size of the layer in Example 5, was required to treat VOC discharged upon influx of benzene.

### INDUSTRIAL APPLICABILITY

Therefore, through the small biofilter system equipped with the load-equalizer in accordance with the present invention, VOC can be stably treated even when VOC is discontinuously generated and the difference of generation concentration is very large. The conventional biofilters can be used only under a stable condition where VOC is continuously generated in the constant ranges of 1000 ppm or less, but the present biofilter system equipped with the VOC load-equalizer allows concentration of VOC to be efficiently controlled in the predetermined ranges. So, under poor discharge conditions, stable activities of microorganisms can be maintained and the system can be designed in small scale. In addition, the biofilter system can obtain stable treatment efficiency even after a long period of operation by the pressurized air/water spray unit mounted to the lower carrier.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A biofilter system for removing volatile organic compounds (VOC) comprising:
a) a load-equalizer comprising a porous carrier layer, an inlet for VOC-containing air positioned under the porous carrier layer, a storage tank for load-equalizing solvent, a circulation unit equipped with a spray nozzle which is connected to the storage tank and sprays the load-equalizing solvent over the carrier layer, said carrier layer being arranged in such a manner that the VOC-containing air contacts with the load-equalizing solvent through the carrier layer to provide the load-equalized air with the VOC concentration range treatable in the subsequent biofiltering by physical transfer of VOC, and an outlet for discharging the load-equalized air;
b) an air transferring portion for introducing the VOC-containing air from VOC generation sources to the load-equalizer and transferring the load-equalized air to a biofiltering portion,
c) a biofiltering portion, comprising a porous microbial carrier layer inhabited by microorganisms for removing VOC, an inlet for the load-equalized air positioned under the porous microbial carrier layer, a medium storage tank and a medium circulation unit, said microbial carrier layer equipped with a pressurized air/water spray unit for shaking and rearranging the microbial carrier layer, thereby suppressing pressure loss and drift of the load-equalized air, said medium circulation unit being connected to the medium storage tank and circulating the medium to two directions by a circulatory pump, in which one direction flows to the biofiltering portion through a upper spray nozzle located above the microbial carrier layer and the other direction flows over the medium through a lower spray nozzle placed under the microbial carrier layer, and
d) a storing portion for adjusting pH of the medium and feeding nutrients, connected to the medium storage tank.

2. The biofilter system as defined in claim 1, further comprising a temperature controller connected to the medium storage tank, thereby maintaining the temperature of the carrier layer on the predetermined level.

3. The biofilter system as defined in claim 2, wherein a heat source of the temperature controller is steam or electricity.

4. The biofilter system as defined in claim 1, further comprising a ring blower for providing air to the lower part of the medium storage tank to facilitate decomposition of VOC by microorganisms present in the medium storage tank.

5. The biofilter system as defined in claim 1, wherein said carrier layer in the load-equalizer is made of at least one material selected from the group consisting of polyethylene, polypropylene, polyester and ceramic.

6. The biofilter system as defined in claim 1, wherein the biofiltering portion further comprises a packing layer mounted below the lower spray nozzle to maximize the contact area of the load-equalized air and the sprayed medium.

7. The biofilter system as defined in claim 6, wherein said packing layer is charged with pall rings in the form of porous cylinders having a diameter and a height of 0.5-2.0 inches, respectively, and made of polyethylene or polypropylene.

8. The biofilter system as defined in claim 1, wherein said carrier layer in the load-equalizer is structured in two or more stages.

9. The biofilter system as defined in claim 1, wherein the volume of said carrier layer in the load-equalizer corresponds to 10-50% of volume of the load-equalizing solvent.

10. The biofilter system as defined in claim 1, wherein said load-equalizing solvent is a lipid-soluble solvent, water-soluble solvent or the combination thereof, the lipid-soluble solvent comprising C₁₄₋₁₅ paraffin and naphthene hydrocarbons, which is a colorless and odorless liquid, without any ozone layer-damaging components, and is non-aromatic solvent having 0.8 to 0.9 g/cm³ specific gravity, 270 to 320° C. boiling point, 110 to 140° C. ignition point and aromatic components of below 0.5%, or comprising silicon oil having a specific gravity of 0.76 to 1.00 g/cm³, viscosity of 0.65 to 10,000 cSt (25° C.), flow point of −75 to 40° C., and surface tension of 20.0 to 25.5 dyne/cm, the water-soluble solvent comprising water.

11. The biofilter system as defined in claim 10, wherein the lipid-soluble solvent has load-equalizing capability constant k of 0.005-0.6 for single benzene ring compounds, and the water-soluble solvent has load-equalizing capability constant k of 0.001-0.1 for water-soluble VOC.

12. The biofilter system as defined in claim 1, wherein the microbial carrier layer of the biofiltering portion is made of at least one material selected from the group consisting of polyether, polyester and polyethylene.

13. The biofilter system as defined in claim 1, wherein the microorganisms used in the microbial carrier layer are at least one selected from the group consisting of Pseudomonas, Aerobacter, Bacillus, Microbacterium and Arthrobacter Sp.

14. The biofilter system as defined in claim 1, wherein said biofiltering portion further comprising a compressor and a controller for controlling the spray unit, whereby the pressurized air/water to be automatically sprayed when pressure loss on introduction of the load-equalized air to the microbial carrier layer reaches a predetermined value.

15. The biofilter system as defined in claim 1, wherein a stream circulating to the medium by the circulatory pump in said medium circulation unit is supplied to the medium opposite the inlet of said circulatory pump.

16. The biofilter system as defined in claim 1, wherein the medium storage tank is mounted below the inlet for the load-equalized air, and corresponds to 10-30% of the volume of the porous microbial carrier layer.

17. The biofilter system as defined in claim 1, wherein the spray nozzle in the medium circulation unit is operated according to the controllable time period with use of a solenoid valve.

18. The biofilter system as defined in claim 1, wherein the pressurized air/water spray unit is located at the lower part of the porous microbial carrier layer to spray the pressurized air/water upwards.

19. The biofilter system as defined in claim 1, wherein the storing portion comprises a pH adjusting solution storage tank for adjusting pH of the medium in the medium storage tank and a high concentration nutrient storage tank for providing the nutrients.
